(19) United States
(12) Patent Application Publication
(10) Pub. No.: US 2016/0353998 A1
(43) Pub. Date: Dec. 8, 2016

(54) APPARATUS AND METHOD FOR NONINVASIVE AND CUFFLESS BLOOD PRESSURE MEASUREMENT

(71) Applicant: National Central University, Jhongli City (TW)

(72) Inventors: Po-Lei Lee, Jhongli City (TW); Kuo-Kai Shyu, Jhongli City (TW); Hao-Teng Shu, Jhongli City (TW); Te-Min Lee, Jhongli City (TW)

(21) Appl. No.: 15/165,542

(22) Filed: May 26, 2016

(30) Foreign Application Priority Data
Jun. 2, 2015 (TW) 104117761

(51) Int. Cl. A61B 5/021; A61B 5/0402; A61B 5/04; A61B 5/00

(52) U.S. Cl. CPC A61B 5/02108; A61B 5/742; A61B 5/7282; A61B 5/0402; A61B 5/04012; A61B 5/681; A61B 5/02433

(57) ABSTRACT

An apparatus and a method for noninvasive and caffless blood pressure measurement are provided. The apparatus includes: a photoplethysmography (PPG) amplitude calculation unit, a time difference calculation unit, a velocity calculation unit, and a calculation unit. The method includes the following steps: calculating amplitude differences between characteristic points of a PPG waveform; calculating differences between a time point of a peak of an electrocardiography (ECG) waveform and a time point of each of the characteristic points of the PPG waveform; calculating differences between time points of the characteristic points of the PPG waveform; performing calculation according to the length of a hand and an ECG-PPG time difference to obtain a pulse wave velocity (PWV);

and performing calculation according to the PWV, one of the characteristic point amplitude difference, and one of the characteristic point time difference to obtain a systolic/diastolic blood pressure estimate.

### BACKGROUND

1. Technical Field

The present invention relates to the field of medical equipment, and in particular, to an apparatus and a method for noninvasive and cuffless blood pressure measurement.

2. Related Art

With the rapid development of industry and commerce, work pressure of people is growing, coupled with an unbalanced diet, resulting in an increasing number of people suffering from cardiovascular diseases. According to statistics of top ten leading causes of death in Taiwan collected by Ministry of Health and Welfare of Executive Yuan from 2008 to 2012, nearly a half of the top ten leading causes of death are cardiovascular related diseases, such as heart disease, cerebrovascular disease, and hypertensive disease. The number of deaths resulted from cardiovascular related diseases is 30.3% of the total number of deaths in Taiwan in 2012, and the heart disease and the cerebrovascular disease are two main causes of the deaths resulted from cardiovascular related diseases.

Many patients consult doctors and have physical examinations only when conditions are serious, not to mention that people cannot detect their diseases by means of physical examinations because of a severe shortage of medical resources in many districts. Therefore, how to implement a self-examination at home rather than an examination in hospital and implement an early detection of a cardiovascular related disease is an important subject.

A traditional apparatus for blood pressure measurement in the market needs to use a sphygmomanometer and use a cuff to apply pressure to measure a blood pressure estimate, and the sphygmomanometer needs to be periodically calibrated, so as to prevent an error value from being generated. Some studies use electrocardiography (ECG) and photoplethysmography (PPG) to perform noninvasive blood pressure calculation, for example, blood pressure calculation is performed by using a pulse wave velocity (PWV) calculated by using ECG and PPG signals and adding a compensatory pressure device to a finger or a wrist. In addition, a PWV calculated by using ECG and PPG is used to perform noninvasive and cuffless blood pressure calculation.

### SUMMARY

In view of the foregoing problem, an objective of the present invention is to provide an apparatus and a method for noninvasive and cuffless blood pressure measurement for measuring physiological signals of a tested person, such as a PPG waveform and an ECG waveform, using a PPG amplitude to represent intravascular blood volume, and using a difference between time points of characteristic points of the PPG waveform as a calibration parameter of a vascular elastic coefficient, so as to calculate an intravascular systolic/diastolic blood pressure. The apparatus for noninvasive and cuffless blood pressure measurement does not need periodic calibration, does not need a cuff for applying pressure, can measure a blood pressure estimate of a tested person according to physiological signals, can be worn by a measured person for a long time, and can measure physiological signals.

A first aspect of the present invention provides an apparatus for noninvasive and cuffless blood pressure measurement, including:
a PPG amplitude calculation unit, configured to calculate amplitude differences between a plurality of characteristic points of a PPG waveform, so as to obtain a plurality of characteristic point amplitude differences;
a time difference calculation unit, configured to calculate differences between a time point of a peak of an ECG waveform and a time point of each of the characteristic points of the PPG waveform, so as to obtain a plurality of ECG-PPG time differences, and configured to calculate differences between time points of the characteristic points of the PPG waveform, so as to obtain a plurality of characteristic point time differences;
a velocity calculation unit, configured to perform calculation according to the length of a hand and one of the ECG-PPG time differences calculated by the time difference calculation unit, so as to obtain a pulse wave velocity (PWV); and
a calculation unit, configured to perform calculation according to the PWV calculated by the velocity calculation unit, one of the characteristic point amplitude differences calculated by the PPG amplitude calculation unit, and one of the characteristic point time differences calculated by the time difference calculation unit, so as to obtain a systolic/diastolic blood pressure estimate.

A second aspect of the present invention provides a method for noninvasive and cuffless blood pressure measurement, including the following steps:
calculating amplitude differences between a plurality of characteristic points of a PPG waveform, so as to obtain a plurality of characteristic point amplitude differences;
calculating differences between a time point of a peak of an ECG waveform and a time point of each of the characteristic points of the PPG waveform, so as to obtain a plurality of ECG-PPG time differences, and calculating differences between time points of the characteristic points of the PPG waveform, so as to obtain a plurality of characteristic point time differences;
performing calculation according to the length of a hand and one of the ECG-PPG time differences, so as to obtain a PWV; and
performing calculation according to the PWV, one of the characteristic point amplitude differences, and one of the characteristic point time differences, so as to obtain a systolic/diastolic blood pressure estimate.

### BRIEF DESCRIPTION OF THE DRAWINGS

FIG. 1A is a front view of a wrist-worn apparatus for noninvasive and cuffless blood pressure measurement according to the present invention;

FIG. 1B is a rear view of the wrist-worn apparatus for noninvasive and cuffless blood pressure measurement according to the present invention;

FIG. 2 is a block diagram of the apparatus for noninvasive and cuffless blood pressure measurement according to the present invention;

FIG. 3 is a diagram of an ECG waveform according to the present invention;

FIG. 4 is a diagram of a PPG waveform according to the present invention;

FIG. 5 is a schematic diagram of characteristic points of the PPG waveform according to the present invention;

FIG. 6 is a schematic diagram of the ECG waveform according to the present invention;

FIG. 7 is a schematic diagram of time differences between time points of peaks of the ECG waveform and a time point of a characteristic point A of the PPG waveform according to the present invention;

FIG. 8 is a schematic diagram of values of amplitude differences between characteristic points of the PPG waveform according to the present invention; and

FIG. 9 is a flowchart of a method for noninvasive and cuffless blood pressure measurement according to the present invention.

### DETAILED DESCRIPTION

To make persons of ordinary skill in the art further understand the present invention, features and effects of the present invention are described in detail below through preferred embodiments with reference to the accompanied drawings.

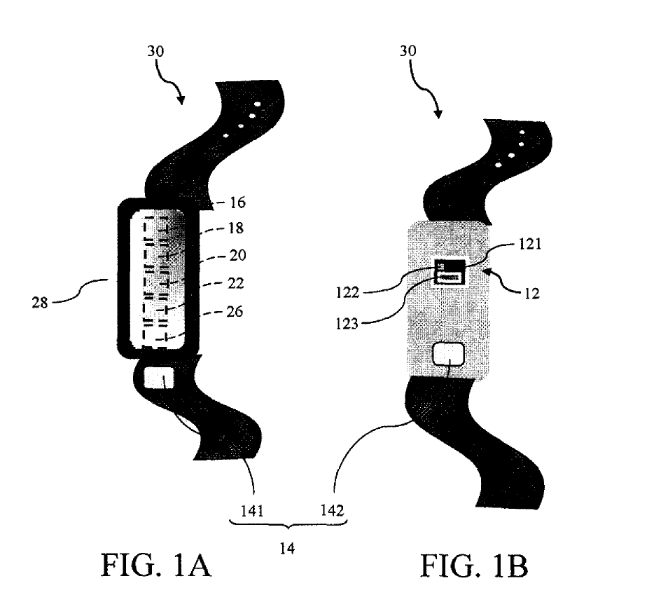

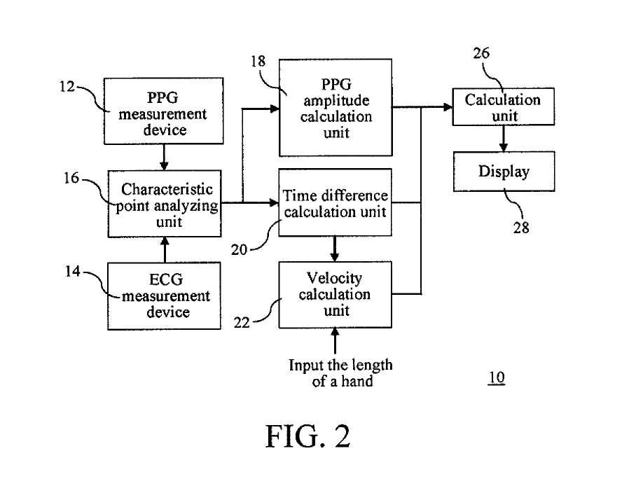

FIG. 1A is a front view of a wrist-worn apparatus for noninvasive and cuffless blood pressure measurement according to the present invention; FIG. 1B is a rear view of the wrist-worn apparatus for noninvasive and cuffless blood pressure measurement according to the present invention; and FIG. 2 is a block diagram of the apparatus for noninvasive and cuffless blood pressure measurement according to the present invention. In FIG. 1A, FIG. 1B, and FIG. 2, an apparatus for noninvasive and cuffless blood pressure measurement 10 includes a PPG measurement device 12, an ECG measurement device 14, a characteristic point analyzing unit 16, a PPG amplitude calculation unit 18, a time difference calculation unit 20, a velocity calculation unit 22, a calculation unit 26, and a display 28.

In this embodiment, the apparatus for noninvasive and cuffless blood pressure measurement 10 is arranged in a wrist-worn member 30 in a wrist-worn manner, but the present invention is not limited thereto. The apparatus for noninvasive and cuffless blood pressure measurement 10 may also be arranged in a finger-worn manner or a head-mounted manner.

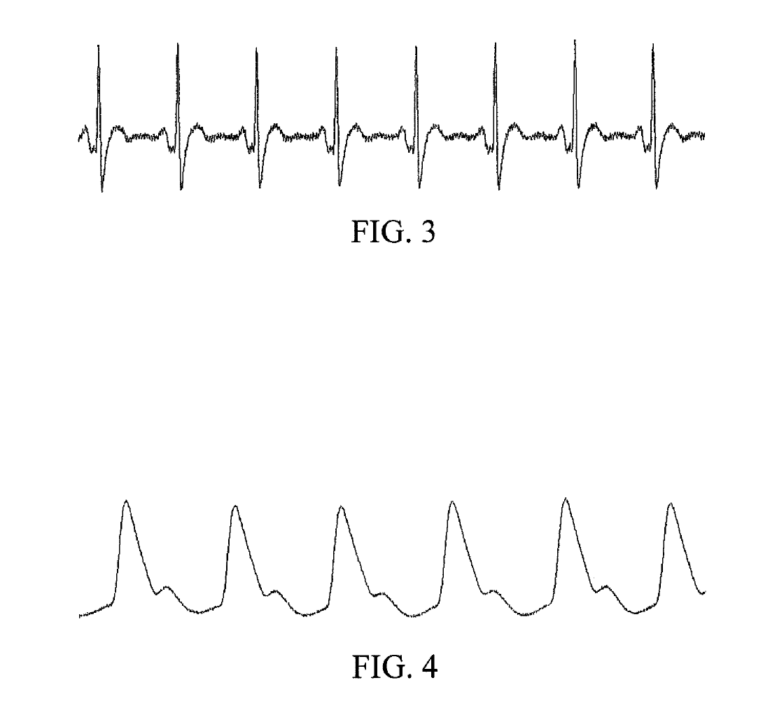

The ECG measurement device 14 includes a first pole piece 141 and a second pole piece 142. The first pole piece 141 is arranged at a front surface of the wrist-worn member 30, and the second pole piece 142 is arranged at a rear surface of the wrist-worn member 30. The first pole piece 141 and the second pole piece 142 are, for example, separately in contact with skin of two hands, so as to measure an ECG waveform of heartbeats as shown in FIG. 3, a diagram of an ECG waveform according to the present invention.

The PPG measurement device 12 arranged at the rear surface of the wrist-worn member 30 includes a first emitter 121, a second emitter 122, and a receiver 123. Each of the first emitter 121 and the second emitter 122 is a transmissive emitter or a reflective emitter for emitting green light, red light, or infrared rays. The receiver 123 is a transmissive receiver or a reflective receiver for receiving green light, red light, or infrared rays. A wavelength of the green light is 495 nm to 570 nm, a wavelength of the red light is 620 nm to 750 nm, and a wavelength of the infrared rays is 780 nm to 1000 nm.

In this embodiment, the PPG measurement device 12 including two emitters is described, but the present invention is not limited thereto. Any specific number of emitters is applicable to the present invention.

The first emitter 121 and the second emitter 122 emit light of the foregoing specific wavelength to an object such as an arm or a finger, and the receiver 123 receives light of a specific wavelength that transmits the finger or the arm or is reflected by the finger or the arm, so that the PPG measurement device 12 measures a PPG waveform indicating blood volume changes under light irradiation, as shown in FIG. 4, a diagram of a PPG waveform according to the present invention.

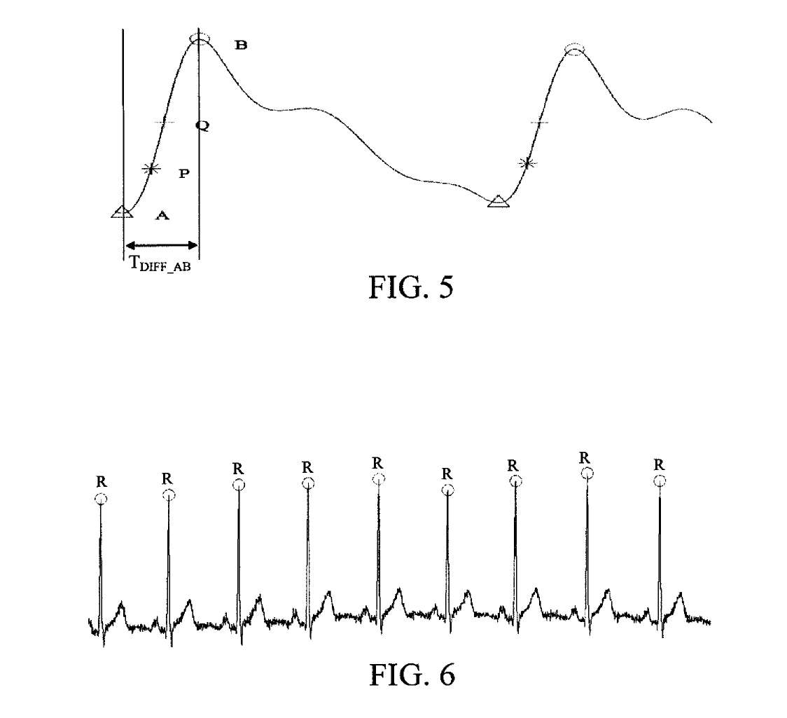

The characteristic point analyzing unit 16 arranged in the wrist-worn member 30 analyzes the PPG waveform measured by the PPG measurement device 12, and refers to the content in Characters available in photoplethysmogram for blood pressure estimation: beyond the pulse transit time (Australasian College Physical Scientists and Engineers in Medicine (2014) 37:367-376 published by Yanjun Li, Zengli Wang, Lin Zhang, Xianglin Yang, Jinzhong Song, etc., so as to obtain amplitudes and time points of characteristics of the PPG waveform as shown in FIG. 5, a schematic diagram of characteristic points of the PPG waveform according to the present invention.

In FIG. 5, a characteristic point B is a peak in the PPG waveform; a characteristic point A is a valley in the PPG waveform; a characteristic point P is a position between the valley (that is, the characteristic point A) and the peak (that is, the characteristic point B) and with a distance between the position and the valley being ¼ of a distance between the valley and the peak; and a characteristic point Q is a position of a maximum slope between the valley and the peak.

The characteristic point analyzing unit 16 arranged in the wrist-worn member 30 analyzes an ECG waveform measured by the ECG measurement device 14, so as to obtain amplitudes and time points of peaks of the ECG waveform as shown in FIG. 6, a schematic diagram of the ECG waveform according to the present invention. In FIG. 6, Rs represent peaks of the ECG waveform.

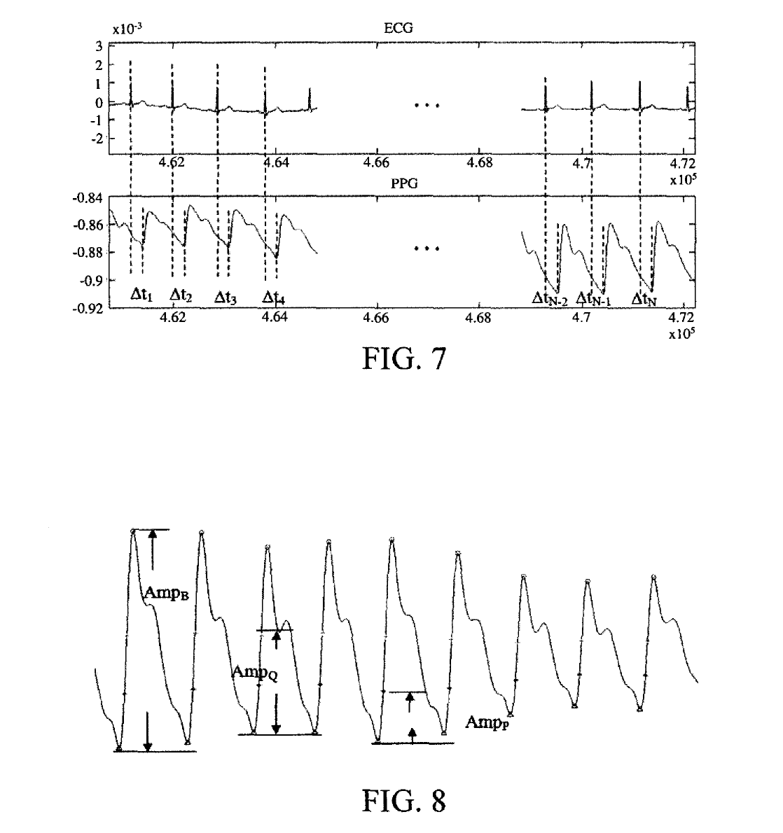

The time difference calculation unit 20 arranged in the wrist worn member 30 calculates differences between time points of the Rs of the ECG waveform and a time point of each of the characteristic points B, Q, P, and A of the PPG waveform, so as to obtain a plurality of ECG-PPG time differences Δt_{N}, for example, as shown in FIG. 7, a schematic diagram of time differences between time points of peaks of the ECG waveform and a time point of the characteristic point A of the PPG waveform according to the present invention, the time difference calculation unit 20 calculates differences between the time points of the Rs of the ECG waveform and the time point of the characteristic point A of the PPG waveform, so as to obtain ECG-PPG time differences Δt_{N}.

The time difference calculation unit 20 calculates differences between time points of the characteristic points B, Q, P, and A of the PPG waveform, so as to obtain a plurality of characteristic point time differences T_{DIFF}, referring to FIG. 5, for example, calculates a difference between a time point of the characteristic point B and a time point of the characteristic point A of the PPG waveform to obtain a characteristic point time difference T_{DIFF}_{_}_{AB}, calculates a difference between a time point of the characteristic point Q and the time point of the characteristic point A of the PPG waveform to obtain a characteristic point time difference T_{DIFF}_{_}_{AQ}, calculates a difference between a time point of the characteristic point P and the time point of the characteristic point A of the PPG waveform to obtain a characteristic point time difference T_{DIFF}_{_}_{AP}, and the like.

The velocity calculation unit 22 arranged in the wrist-worn member 30 performs calculation according to the length of a hand of a tested person and one of the ECG-PPG time differences Δt_{N} calculated by the time difference calculation unit 20, so as to obtain a PWV, where data of the length of the hand of the tested person may be input into the velocity calculation unit 22 by an input apparatus (not shown) of the wrist-worn member 30.

The PPG amplitude calculation unit 18 arranged in the wrist-worn member 30 calculates amplitude differences between the characteristic points B, Q, P, and A of the PPG waveform to obtain a plurality of characteristic point amplitude differences Amp, referring to FIG. 8, a schematic diagram of values of characteristic point amplitude differences of the PPG waveform according to the present invention, for example, calculates an amplitude difference between the characteristic point B and the characteristic point A of the PPG waveform to obtain a characteristic point amplitude difference Amp_{B}, calculates an amplitude difference between the characteristic point Q and the characteristic point A of the PPG waveform to obtain a characteristic point amplitude difference Amp_{Q}, calculates an amplitude difference between the characteristic point P and the characteristic point A of the PPG waveform to obtain a characteristic point amplitude difference Amp_{P}, and the like.

The calculation unit 26 arranged in the wrist-worn member 30 performs calculation according to the PWV calculated by the velocity calculation unit 22, one of the plurality of characteristic point amplitude differences Amp calculated by the PPG amplitude calculation unit 18, and one of the plurality of characteristic point time differences T_{DIFF} calculated by the time difference calculation unit 20, so as to obtain a systolic/diastolic blood pressure estimate BP_{EST}.

The systolic/diastolic blood pressure estimate BP_{EST}=C1*PWV+C2*Amp+C3*T_{DIFF}+C4, where C1, C2, C3, and C4 are constants; the pulse wave velocity PWV=the length of a hand/Δt_{N}; Δt_{N} is an ECG-PPG time difference; Amp is a characteristic point amplitude difference; and T_{DIFF} is a characteristic point time difference. In the present invention, according to a requirement of a systolic/diastolic blood pressure, calculation may be performed by using the foregoing parameters.

A display 28 that may be a liquid crystal display (LCD) screen is arranged at a front surface of the wrist-worn member 30. The display 28 is configured to display the systolic/diastolic blood pressure estimate BP_{EST} calculated by the calculation unit 26, so that a blood pressure estimate may be directly observed since the apparatus can be worn by a tested person for a long time and can measure physiological signals.

Operations of a method for noninvasive and cuffless blood pressure measurement of the present invention are described with reference to the block diagram and the configuration diagram of an apparatus for noninvasive and cuffless blood pressure measurement, the diagrams of waveforms measured by the apparatus for noninvasive and cuffless blood pressure measurement, and other relevant drawings.

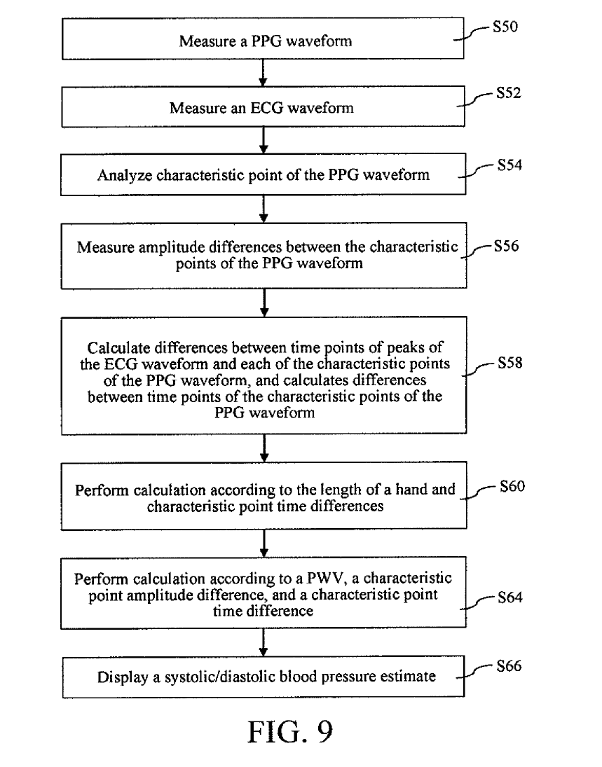

FIG. 9 is a flowchart of a method for noninvasive and cuffless blood pressure measurement according to the present invention. In FIG. 9, a first emitter 121 and a second emitter 122 of a PPG measurement device 12 arranged at a rear surface of a wrist-worn member 30 emit the foregoing light of a specific wavelength (green light, red light, or infrared rays) to an arm or a finger, and a receiver 123 of the PPG measurement device 12 receives light of a specific wavelength that transmits the finger or arm or is reflected by the finger or arm, so that the PPG measurement device 12 measures a PPG waveform indicating blood volume changes under light irradiation (step S50), as shown in the waveform of FIG. 4.

A first pole piece 141 and a second pole piece 142 of an ECG measurement device 14 separately arranged at a front surface and a rear surface of the wrist-worn member 30 are separately in contact with skin of two hands, so as to measure an ECG waveform of heartbeats (step S52), as shown in the waveform of FIG. 3.

A characteristic point analyzing unit 16 arranged in the writs-worn member 30 analyzes the PPG waveform measured by the PPG measurement device 12, and refers to the content of the foregoing document, so as to obtain amplitudes and time points of characteristic points B, Q, P, and A of the PPG waveform (step S54), as shown in FIG. 5.

In FIG. 5, the characteristic point B is a peak in the PPG waveform; the characteristic point A is a valley in the PPG waveform; the characteristic point P is a position between the valley (that is, the characteristic point A) and the peak (that is, the characteristic point B) and with a distance between the position and the valley being ¼ of a distance between the valley and the peak; and the characteristic point Q is a position of a maximum slope between the valley and the peak.

Referring to FIG. 8, a PPG amplitude calculation unit 18 arranged in the wrist-worn member 30 calculates an amplitude difference between the characteristic point B and the characteristic point A of the PPG waveform, so as to obtain a characteristic point amplitude difference Amp_{B}, calculates an amplitude difference between the characteristic point Q and the characteristic point A of the PPG waveform, so as to obtain a characteristic point amplitude difference Amp_{Q}, calculates an amplitude difference between the characteristic point P and the characteristic point A of the PPG waveform, so as to obtain a characteristic point amplitude difference Amp_{P}, and performs calculation to obtain a plurality of characteristic point amplitude differences Amp (step S56).

A time difference calculation unit 20 arranged in the wrist-worn member 30 calculates differences between time points of Rs of the ECG waveform and each of the characteristic points B, Q, P, and A of the PPG waveform, so as to obtain a plurality of ECG-PPG time differences Δt_{N}. As shown in FIG. 7, the time difference calculation unit 20 calculates differences between the time points of the Rs of the ECG waveform and the time point of the characteristic point A of the PPG waveform to obtain ECG-PPG time differences Δt_{N}.

Referring to FIG. 5, the time difference calculation unit 20 calculates a difference between a time point of the characteristic point B and a time point of the characteristic point A of the PPG waveform to obtain a characteristic point time difference T_{DIFF}_{_}_{AB}, calculates a difference between a time point of the characteristic point Q and the time point of the characteristic point A of the PPG waveform to obtain a characteristic point time difference T_{DIFF}_{_}_{AQ}, calculates a difference between a time point of the characteristic point

P and the time point of the characteristic point A of the PPG waveform to obtain a characteristic point time difference T_{DIFF}_{_}_{AN} and performs calculation to obtain a plurality of characteristic point time differences T_{DIFF} (step S58).

A velocity calculation unit 22 arranged in the wrist-worn member 30 performs calculation according to the length of a hand of a tested person, and one of the ECG-PPG time differences Δt_{N} calculated by the time difference calculation unit 20, so as to obtain a PWV (step S60), where data of the length of the hand of the tested person may be input into the velocity calculation unit 22 by an input apparatus (not shown) of the wrist-worn member 30.

By means of the foregoing formula for calculating a blood pressure estimate, a calculation unit 26 arranged in the wrist-worn member 30 performs calculation according to the PWV calculated by the velocity calculation unit 22, one of the plurality of characteristic point amplitude differences Amp calculated by the PPG amplitude calculation unit 18, and one of the plurality of characteristic point time differences T_{DIFF} calculated by the time difference calculation unit 20, so as to obtain a systolic/diastolic blood pressure estimate BP_{EST} (step S64).

A display 28 arranged at the front surface of the wrist-worn member 30 displays the systolic/diastolic blood pressure estimate BP_{EST} calculated by the calculation unit 26, so that a blood pressure estimate can be directly observed since the apparatus can be worn by a tested person for a long time and can measure physiological signals (step S66).

An objective of the present invention is to provide an apparatus and a method for noninvasive and cuffless blood pressure measurement with advantages of measuring physiological signals of a tested person, such as a PPG waveform and an ECG waveform, using a PPG amplitude to represent intravascular blood volume, and using a difference between time points of characteristic points of the PPG waveform as a calibration parameter of a vascular elastic coefficient, so as to calculate an intravascular systolic/diastolic blood pressure. The apparatus for noninvasive and cuffless blood pressure measurement does not need periodic calibration, does not need a cuff for applying pressure, can measure a blood pressure estimate of a tested person according to physiological signals, can be worn by a tested person for a long time, and can measure physiological signals.

The present invention is described above with reference to preferred embodiments and exemplary drawings, but is not limited thereto. Various modifications, omissions, and variations made to the forms and content of the embodiments by persons killed in the art shall not go beyond the scope claimed in the claims of the present invention.

### DESCRIPTION OF SYMBOLS

10 Apparatus for noninvasive and cuffless blood pressure measurement
12 PPG measurement device
14 ECG measurement device
16 Characteristic point analyzing unit
18 PPG amplitude calculation unit
20 Time difference calculation unit
22 Velocity calculation unit
26 Calculation unit
28 Display
30 Wrist-worn member
121 First emitter
122 Second emitter
123 Receiver
141 First pole piece
142 Second pole piece

What is claimed is:

1. An apparatus for noninvasive and cuffless blood pressure measurement, comprising:
a photoplethysmography (PPG) amplitude calculation unit, configured to calculate amplitude differences between a plurality of characteristic points of a PPG waveform, so as to obtain a plurality of characteristic point amplitude differences;
a time difference calculation unit, configured to calculate differences between a time point of a peak of an ECG waveform and a time point of each of the characteristic points of the PPG waveform, so as to obtain a plurality of ECG-PPG time differences, and configured to calculate differences between time points of the characteristic points of the PPG waveform, so as to obtain a plurality of characteristic point time differences;
a velocity calculation unit, configured to perform calculation according to the length of a hand and one of the ECG-PPG time differences calculated by the time difference calculation unit, so as to obtain a pulse wave velocity (PWV); and
a calculation unit, configured to perform calculation according to the PWV calculated by the velocity calculation unit, one of the characteristic point amplitude differences calculated by the PPG amplitude calculation unit, and one of the characteristic point time differences calculated by the time difference calculation unit, so as to obtain a systolic/diastolic blood pressure estimate.

2. The apparatus according to claim 1, further comprising:
a PPG measurement device, comprising: a receiver and at least one emitter, wherein the at least one emitter emits light of a specific wavelength to an object, and the receiver receives light of a specific wavelength that transmits the object or is reflected by the object, so as to measure a PPG waveform indicating blood volume changes under light irradiation;
an ECG measurement device, configured to measure an ECG waveform of heartbeats;
a characteristic point analyzing unit, configured to analyze the PPG waveform measured by the PPG measurement device, so as to obtain amplitudes and time points of the characteristic points of the PPG waveform, and configured to analyze the ECG waveform measured by the ECG measurement device, so as to obtain an amplitude and a time point of the peak of the ECG waveform; and
a display, configured to display the systolic/diastolic blood pressure estimate calculated by the calculation unit.

3. The apparatus according to claim 2, wherein the at least one emitter is a transmissive emitter or a reflective emitter for emitting one of green light, red light, and infrared rays, and the receiver is a transmissive receiver or a reflective receiver for receiving one of the green light, the red light, and the infrared rays.

4. The apparatus according to claim 3, wherein a wavelength of the green light is 495 nm to 570 nm, a wavelength of the red light is 620 nm to 750 nm, and a wavelength of the infrared rays is 780 nm to 1000 nm.

5. The apparatus according to claim 2, wherein the display is a liquid crystal display (LCD) screen.

6. The apparatus according to claim 2, wherein the characteristic points comprise the peak, a valley, a position between the valley and the peak and with a distance between the position and the valley being 1/4 of a distance between the valley and the peak, and a position of a maximum slope between the valley and the peak in the PPG waveform.

7. The apparatus according to claim 6, wherein the systolic/diastolic blood pressure estimate BP_{EST}=C1*PWV+C2*Amp+C3*T_{DIFF}+C4, wherein C1, C2, C3, and C4 are constants; PWV is the pulse wave velocity; PWV=the length of a hand/Δt_{N}; Δt_{N} is an ECG-PPG time difference; Amp is a characteristic point amplitude difference; and T_{DIFF} is a characteristic point time difference.

8. A method for noninvasive and cuffless blood pressure measurement, comprising the following steps:
calculating amplitude differences between a plurality of characteristic points of a PPG waveform, so as to obtain a plurality of characteristic point amplitude differences;
calculating differences between a time point of a peak of an ECG waveform and a time point of each of the characteristic points of the PPG waveform, so as to obtain a plurality of ECG-PPG time differences, and calculating differences between time points of the characteristic points of the PPG waveform, so as to obtain a plurality of characteristic point time differences;
performing calculation according to the length of a hand and one of the ECG-PPG time differences, so as to obtain a PWV; and
performing calculation according to the PWV, one of the characteristic point amplitude differences, and one of the characteristic point time differences, so as to obtain a systolic/diastolic blood pressure estimate.

9. The method according to claim 8, wherein before the calculating characteristic point amplitude differences of a PPG waveform, the method further comprises the following steps:
emitting light of a specific wavelength to an object, and receiving light of a specific wavelength that transmits the object or is reflected by the object, so as to measure a PPG waveform indicating blood volume changes under light irradiation;
measuring an ECG waveform of heartbeats; and
analyzing the PPG waveform to obtain amplitudes and time points of the characteristic points of the PPG waveform, and analyzing the ECG waveform to obtain an amplitude and a time point of the peak of the ECG waveform.

10. The method according to claim 9, wherein after the performing calculation according to the PWV, one of the characteristic point amplitude differences, and one of the characteristic point time differences, the method further comprises the following step:
displaying the systolic/diastolic blood pressure estimate.

11. The method according to claim 9, wherein the light of the specific wavelength is one of green light of a wavelength of 495 nm to 570 rim, red light of a wavelength of 620 nm to 750 nm, and infrared rays of a wavelength of 780 nm to 1000 nm.

12. The method according to claim 9, wherein the characteristic points comprise the peak, a valley, a position between the valley and the peak and with a distance between the position and the valley being ¼ of a distance between the valley and the peak, and a position of a maximum slope between the valley and the peak in the PPG waveform.

13. The apparatus according to claim 9, wherein the systolic/diastolic blood pressure estimate BP_{EST}=C1*PWV+C2*Amp+C3*T_{DIFF}+C4, wherein C1, C2, C3, and C4 are constants; PWV is the pulse wave velocity; PWV=the length of a hand/Δt_{N}; Δt_{N} is an ECG-PPG time difference; Amp is a characteristic point amplitude difference; and T_{DIFF} is a characteristic point time difference.
